(19) United States
(12) Plant Patent Application Publication
(10) Pub. No.: US 2003/0126660 P1
(43) Pub. Date: Jul. 3, 2003

(54) CHRYSANTHEMUM PLANT NAMED 'MN95-105-6'

(76) Inventors: Neil Owen Anderson, St. Paul, MN (US); Peter David Ascher, St. Paul, MN (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 09/503,578

(22) Filed: Feb. 14, 2000

(51) Int. Cl. A01H 5/00

(52) U.S. Cl. PLT/284

(57) ABSTRACT

A new and distinct Chrysanthemum plant named 95-105-6 is provided. This new cultivar was the result of a unique cross between Dendranthema weyrichii and Dendranthema grandiflora.

### BACKGROUND OF THE INVENTION

The present invention comprises a new and distinctive chrysanthemum plant, hereinafter referred to by the cultivar name 95-105-6. This new cultivar was the result of a cross in 1989 between Dendranthema weyrichii and Dendranthema grandiflora. More specifically, the breeding program which resulted in the production of the new cultivar was carried out at St. Paul, Minn. The female or seed parent of 95-105-6 was a Dendranthema weyrichii, commercially available from White Flower Farms, Connecticut having the following characteristics: (a) the plant habit is prostrate and the plant spreads via rhizomes to form a large mat after the first year; (b) the plant dimensions are that the plant has a diameter of about 1.5′ and is about 5-6″ tall; (c) the plant is hardy in zones 4-9 (Southeast)/Zone 10 (west); (d) the flower of the plant is a single daisy, having light lavender-colored ray florets and central disc florets with yellow pollen; (e) the plant has leaves that are dark green in color, with a very shiny leaf surface (glossy), and glabrous leaf margins that are deeply incised; and (d) the plant tends to rosette, needs cold treatment to flower consistently, flowering can be sporadic with gaps in the plant architecture and the plant is an obligate short-day plant. The male or pollen parent of 95-105-6 was a Dendranthema grandiflora which is commercially available from Yoder Brothers, Inc., Barberton, Ohio having the following characteristics: (a) the plant habit is cushion; (b) the plant dimensions are that the plant is similar to other cushion types commercially available from Yoder Brothers, Inc., such as, but not limited to the variety, ‘Soft Cherie’; (d) the plant is hardy in zones 6-9 (Southeast)/Zone 10 (west); (d) the flower is a single or duplex daisy, possibly orange or bronze ray florets, central disc florets with yellow pollen; (e) the plant has leaves that are similar to other Yoder Brother, Inc. cushion series chrysanthemums; and (d) the plant is a facultative short-day plant. The resulting seeds, identified as 90-287-158 were collected. In 1990, a plant of 90-287-158 was crossed as the male parent with plants identified as 88-409-33, a University of Minnesota variety named “Rose Blush’, as the female parent and the resulting seeds, identified as cross number 91-204-2 were collected. In 1993, seedlings of the cross 91-204-2 were selfed and the resulting seeds, identified as 94-23-87 collected. In 1994, plants of 94-23-87 were selfed and the resulting seeds, identified as cross number 95-105 were collected. In 1995, seedlings of cross 95-105 were germinated and flowering progeny evaluated. 95-105-6 was the sixth plant from the cross and was selected during the fall of 1995. The parentage of the new cultivar can be summarized as follows:

Dendranthema weyrichii×Dendranthema grandiflora

Asexual reproduction of the new cultivar by terminal or stem cuttings taken during 1996 through 1999 at St. Paul, Minn., U.S.A. has demonstrated that the characteristics of the new cultivar as herein described are firmly fixed and are retained through successive generations of such asexual propagation.

### SUMMARY OF INVENTION

It was found that the cultivar of the present invention:

(a) exhibits extreme hybrid vigor,

(b) develops, in its second and subsequent years after planting, when grown in the fall under natural daylength and without the application of growth regulators, into a flowering herbaceous shrub having a plant height of from about 1.9 to about 2.4 feet and a spread from about 2.5 to about 6.0 feet,

(c) exhibits, in its second and subsequent years after planting and during the fall season (August-October), a massive floral display,

(d) displays flowers which are slightly toned with grey, giving the flower petals a slightly altered coloration,

(e) exhibits superior winter hardiness, including frost tolerance, and

(f) exhibits self-pinching.

The 95-105-6 cultivar has not been observed under all possible environmental conditions to date. Accordingly, it is possible that the phenotype may vary somewhat with variations in the environment, such as temperature, light intensity, and day length.

When the new cultivar of the present invention is compared to ‘Jennifer’ (U.S. Plant Pat. No. 8,987), it is found to exhibit a more spreading and prolific habit in its second and subsequent years after planting accompanied with a massive floral display. Reference is made to attached Chart A which compares certain characteristics of 95-105-6 to ‘Jennifer’.

### BRIEF DESCRIPTION OF THE PHOTOGRAPHS

The accompanying photographs show as nearly true as it is reasonably possible to make the same in color illustrations of this type, typical flower and foliage characteristics of the new cultivar. The plants were grown in a greenhouse at St. Paul, Minn., USA.

FIG. 1 shows an adaxial and abaxial views of the leaf shape of chrysanthemum variety 95-105-6.

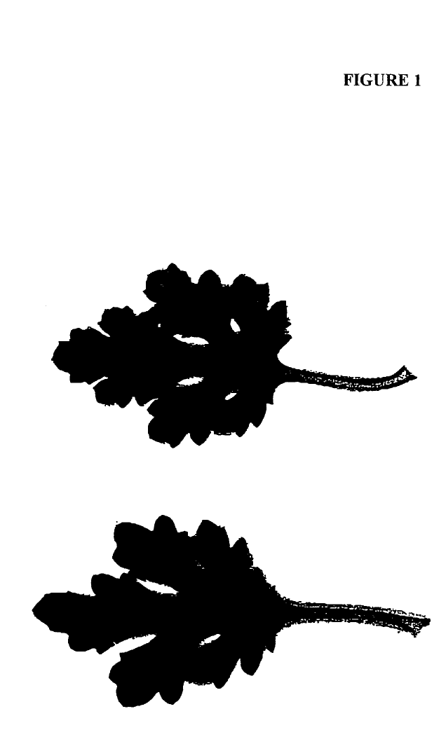

FIG. 2 shows the breeding history of chrysanthemum variety 95-105-6

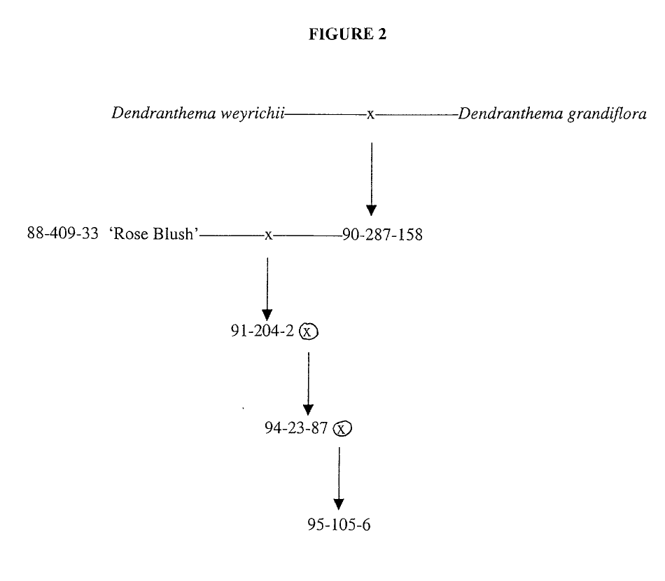

FIG. 3 is a color photograph of chrysanthemum variety 95-105-6 after one year of growth.

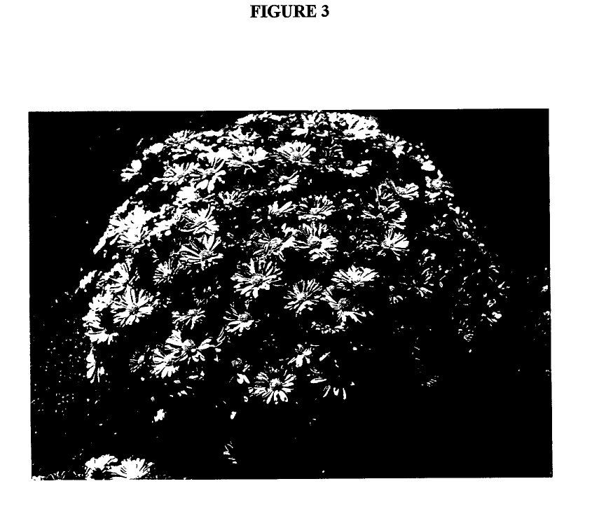

FIG. 4 is a color photograph of chrysanthemum variety 95-105-6 after two years of growth.

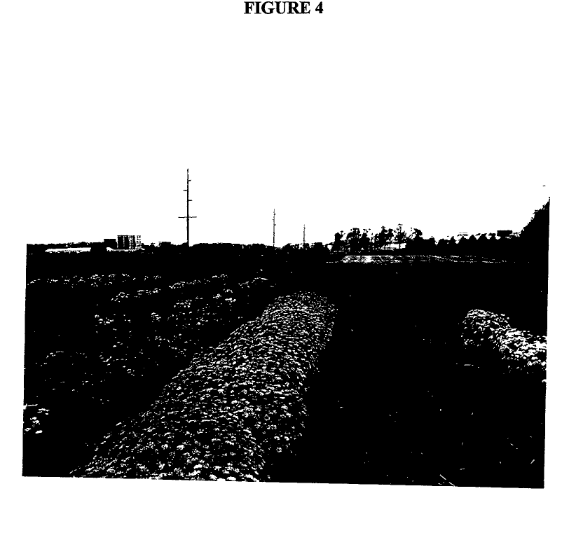

### DETAILED BOTANICAL DESCRIPTION

The chart used in the identification of colors described herein is the R.H.S. Colour Chart of The Royal Horticultural Society, London, England. The color values were determined on Oct. 15, 1999 in St. Paul, Minn. The readings were taken between 1:00 and 2:00 p.m. under approximately 2500 footcandles of light. The plants were produced from cuttings taken from stock plants and were grown under greenhouse conditions comparable to those used in commercial practice while utilizing a soilless growth medium and maintaining temperatures of approximately 72° F. during the day and approximately 65° F. during the night.

Propagation:

Type.—Herbaceous stem cutting.

Time to rooting.—About 1 week.

Rooting habit.—Vigorous.

Botanical Classification: Dendranthema×hybrida

Commercial Classification: Chrysanthemum hybrid

Plant Description:

Appearance, shape.—Flattened mound (first year) Spherical mound (second year).

Appearance, growth habit.—Mound.

Appearance, growth rate/vigor.—Vigorous.

Plant height.—about 14 to about 19 inches (first year) about 1.9 to about 2.4 feet (second year).

Lateral branch length.—1 to 3 feet.

Quantity of lateral branches after removal of apical meristem.—One per node.

Stem color.—RHS Scheeles Green 860/3.

Foliage Description:

Number of leaves per plant.—Greater than 10,000 (second year).

Number of leaves per lateral branch.—5 to 25.

Leaf arrangement.—Alternate.

Leaf size, fully expanded, length.—9.7 cm.

Leaf size, fully expanded, width.—4.6 cm.

Leaf apex.—Obtuse to mucronate.

Leaf base.—Cuneate to oblique.

Leaf margin.—Incised (Mulberry-like incisions).

Leaf texture.—Glabrous.

Petiole length.—3.8 cm.

Color, young foliage adaxial surface.—RHS Scheeles Green 860.

Color, young foliage abaxial surface.—RHS Spinach Green o960/3.

Color, fully expanded foliage adaxial surface.—RHS Ivy Green ooo1060/3.

Color, fully expanded foliage abaxial surface.—RHS Parsley Green oo962/2.

Color, venation adaxial surface.—RHS Lavender Green ooo761/1.

Color, venation abaxial surface.—RHS Lavender Green ooo761/2.

Color, petiole.—RHS Spinach Green o960.

Inflorescence Description:

Appearance.—Head (composite), triplex-quadriplex daisy.

Flowering response.—About 5.5 weeks (SD).

Quantity of inflorescences.—About 800 (first year) About 6,000 (second year).

Inflorescence size, diameter.—6.3 cm.

Inflorescence size, depth (height).—2.3 cm.

Inflorescence size, diameter of disc.—1.5 cm.

Opening inflorescences, bud shape.—Flattened hemisphere to widened upright tubular.

Opening inflorescences, bud size, length.—0.7 cm.

Opening inflorescences, bud size, width.—0.9 cm.

Opening inflorescences, bud color.—RHS Pansy Purple 928/1.

Ray florets, shape.—Linear lanceolate.

Ray florets, size, length.—3 cm.

Ray florets, size, width.—0.7 cm.

Ray florets, apex.—Multi-notched mucronulate.

Ray florets, base.—Attenuate.

Ray florets, margin.—Entire.

Ray florets, texture.—Glabrous.

Ray florets, aspect.—From about 45° vertical to slightly pendant 45°.

Number of ray florets per inflorescence.—About 60.

Ray florets, color, when opening, adaxial surface.—RHS Garnet Lake 828.

Ray florets, color, when opening, abaxial surface.—RHS Chrysanthemum Crimson 824.

Ray florets, color, mature, adaxial surface.—RHS Indian Lake 826/2.

Ray florets, color, mature, abaxial surface.—RHS Erythrite Red oo27/2.

Ray florets, color, fading to.—RHS Erythrite Red oo27/3.

Disc florets, shape.—Tubular, rounded at tip.

Disc florets, size, length.—0.6 cm.

Disc florets, size, width.—0.4 cm freeze usually takes place between October 1-20).

Number of disc florets per inflorescence.—About 191.

Disc florets, color, immature.—RHS Straw Yellow 604.

Disc florets, color, mature.—RHS Maize Yellow 607/1 with an eye in the center of each disc which is RHS Oxblood Red oo823.

Peduncle, aspect, strength.—Stiff.

Peduncle, aspect, angle to stem.—30°.

Peduncle, length, first peduncle.—4.2 cm.

Peduncle, length, fourth peduncle.—7 cm.

Peduncle, texture.—Slightly hirsute.

Peduncle, color.—RHS Spinach Green o960/2.

Reproductive organs, androecium, floret location.—Disc florets.

Anther color.—RHS Chinese Yellow 606/1.

Pollen, abundance.—Abundant.

Pollen, color.—RHS Buttercup Yellow 5.

Reproductive organs, gynoecium, floret location.—Disc/ray florets.

Style color.—RHS Egyptian Buff 407/1.

Disease Resistance: None Known

Seed Production: About 251 ovules/flower

Winter Hardiness: Hardy in zones 3-10 in uncovered field conditions without the need for added protection such as snow fences, mulch, etc,

Frost Tolerance: Yes, extends blooming season to the first freeze in the north (In zones 3-4 the first frost usually takes place between September 1-15. In zones 3-4, the first

What is claimed is:

1. A new and distinct cultivar of Chrysanthemum named 95-105-6 substantially as herein shown and described, which:
(a) exhibits extreme hybrid vigor,
(b) develops, in its second and subsequent years after planting, when grown in the fall under natural daylength without the application of growth regulators, into a flowering herbaceous shrub having a plant height of from about 1.9 to about 2.4 feet and a spread from about 2.5 to about 6.0 feet,
(c) exhibits, in its second and subsequent years after planting and during the fall season (August-October), a massive floral display,
(d) displays flowers which are slightly toned with grey, giving the flower petals a slightly altered coloration,
(e) exhibits superior winter hardiness, including frost tolerance, and
(f) exhibits self-pinching.

| Characteristic | 95-105-6 | ‘Jennifer’ |
|---|---|---|
| Capitulum form and type | Triplex-quadriplex daisy | Flat decorative |
| Plant Height | About 14 to 19 inches | 12 to 14 inches |
| | (first year); about | |
| | 2.4 to about 2.9 feet | |
| | (second year) | |
| Branching Pattern | Spreading and very | Spreading |
| | prolific | |
| Flowering Response | 5.5 weeks | 7 weeks |
| Flower diameter | 6.3 cm | 6.4 to 7.3 cm |
| Ray florets, color, mature | Coral | Bronze with darker |
| | | center |
